(19) United States
(12) Patent Application Publication
(10) Pub. No.: US 2009/0311191 A1
(43) Pub. Date: Dec. 17, 2009

(54) IMAGING OF ATHEROSCLEROTIC PLAQUES USING LIPOSOMAL IMAGING AGENTS

(76) Inventors: Ananth Annapragada, Manvel, TX (US); Russell M. Lebovitz, Houston, TX (US); Devadatta V. Tata, Houston, TX (US); Ketankumar B. Ghaghada, Houston, TX (US); Rohan Bhavane, Houston, TX (US)

Correspondence Address: BENESCH, FRIEDLANDER, COPLAN & ARONOFF LLP;ATTN: IP DEPARTMENT DOCKET CLERK, 200 PUBLIC SQUARE, SUITE 2300, CLEVELAND, OH, 44114-2378, US

(21) Appl. No.: 12/483,758

(22) Filed: Jun. 12, 2009

Related U.S. Application Data
(60) Provisional application No. 61/061,342, filed on Jun. 13, 2008.

(51) Int. Cl. A61K 49/18; A61K 49/00; A61K 49/04; A61K 49/22

(52) U.S. Cl. 424/9.3; 424/9.1; 424/9.4; 424/9.51

(57) ABSTRACT

Compositions and methods are disclosed for imaging atherosclerotic plaques. Example compositions comprise liposomes, the liposomes comprising: at least one first lipid or phospholipid; at least one second lipid or phospholipid which is derivatized with one or more polymers; and at least one sterically bulky excipient capable of stabilizing the liposomes. The liposomes encapsulate or associate a contrast enhancing agent.

### CROSS-REFERENCE TO RELATED APPLICATIONS

This application claims priority from U.S. Provisional Patent Application No. 61/061,342, filed on Jun. 13, 2008, which is incorporated by reference herein in its entirety.

### BACKGROUND

Coronary heart disease is the leading cause of death in the United States for men and women. Many factors exist that increase the risk for coronary heart disease. Some of the risks are based on family history (i.e., genetics). Other risk factors include male gender, age, tobacco use, high blood pressure, diabetes, cholesterol levels (specifically, high low-density lipoprotein cholesterol levels and low high-density lipoprotein cholesterol levels), lack of physical activity, obesity, high blood homocysteine levels, and post-menopause in women. Still other factors include inflammatory responses within an arterial wall. Activation of macrophages (phagocytic white blood cells involved in the removal of foreign material from within body tissues) located in the inner walls of the coronary arteries may play a role in the formation of coronary plaques. Macrophages can migrate to areas of inflammation and foreign material deposits, such as vascular plaques.

Coronary heart disease is characterized by the narrowing of the small blood vessels that supply blood and oxygen to the heart. Coronary heart disease usually results from the build up of fatty material and plaque (atherosclerosis). The buildup is often associated with fibrous connective tissue and frequently includes deposits of calcium salts and other residual material. The damage caused by coronary heart disease varies. As the arteries narrow, the flow of blood to the heart can slow or stop, resulting in symptoms such as chest pains (stable angina), shortness of breath, or a heart attack (i.e. myocardial infarction). Thrombus formation may also result in areas roughened by plaque build-up.

“Vulnerable” or “active” plaque has a tendency to rupture under hemostatic pressure and is, thus, highly susceptible to rapid formation of thrombi leading to acute myocardial infarct (MI) or stroke. Vulnerable plaques thus represent likely sites for future acute cardiovascular events leading to MI or stroke. However, vulnerable plaques are currently difficult to detect using conventional radiological methods and angiography due to the relative absence of calcification in these plaques. Relief of focal high-grade obstruction may control symptoms, but the patient usually is left with numerous non-obstructive plaques prone to later rupture.

Imaging and detection of coronary atherosclerosis and vascular imaging using intravenous contrast medium enhancement is currently available. However, these methods and media are dependent on many complex factors, including the type of media, volume, concentration, injection technique, catheter size and site, imaging technique, cardiac output, and tissue characteristics. Only some of these factors are controllable by radiologists. For example, mixing or streak artifacts can compromise interpretation of computed tomography scans of the abdomen. These artifacts are primarily related to the first pass (arterial phase) effects of intravenous contrast on vascular enhancement. Diffusion of contrast media outside the vascular space not only degrades lesion conspicuity, but also requires that imaging be performed within only a few minutes after the start of injection. Very rapid elimination through the kidneys renders these substances unsuitable for imaging of the vascular system since they cannot provide acceptable contrasts for a sufficient time. All of these difficulties are accentuated in indications that require a consistent contrast enhancement of the vascular blood pool in various vascular beds. Accordingly, improved imaging methods and imaging agents will have broad clinical utility.

### SUMMARY

In one embodiment, a method for imaging atherosclerotic plaques is provided, the method comprising: introducing a composition into a subject's vasculature, the composition comprising: liposomes, the liposomes encapsulating one or more nonradioactive contrast-enhancing agents, and the liposomes comprising: cholesterol, at least one phospholipid, and at least one phospholipid which is derivatized with a polymer chain, wherein the average diameter of the liposomes is less than 150 nanometers; generating images of the subject's vasculature; and analyzing the images to detect and/or evaluate an atherosclerotic plaque in the subject.

In another embodiment, a method for imaging atherosclerotic plaques in a human subject is provided, the method comprising: administering a liposomal composition comprising liposomes to the human subject, the liposomes comprising: at least one first lipid or phospholipid; at least one second lipid or phospholipid which is derivatized with one or more polymers; and at least one sterically bulky excipient capable of stabilizing the liposomes; and wherein the liposomes: (1) encapsulate a non-radioactive contrast enhancing agent in a concentration of about 130-200 mg of non-radioactive contrast enhancing agent per mL of liposomal composition; and (2) have an average diameter of less than 150 nm.

### BRIEF DESCRIPTION OF THE DRAWINGS

The accompanying figures, which are incorporated in and constitute a part of the specification, illustrate various example compositions, methods, results, and so on, and are used merely to illustrate various example embodiments.

FIG. 1 shows representative fluorescence microscopy images of plaque sections obtained from an ApoE −/− mouse that was injected with fluorescein iso-thiocynate (FITC)-encapsulated liposomes. Image A demonstrates the staining of macrophages for F4/80 antigen (darkened regions); the cell nucleus is counterstained with hematoxylin. Image B demonstrates the co-localization of FITC-encapsulated liposomes (bright spots) with the macrophages (arrows) in the plaque. Image C is the corresponding bright-field image.

FIG. 2 shows representative fluorescence microscopy images of plaque sections obtained from an ApoE −/− mouse that was injected with FITC-encapsulated liposomes. Image A demonstrates the staining of macrophages for F4/80 antigen (darkened region); the cell nucleus is counterstained with hematoxylin. Image B demonstrates the co-localization of FITC-encapsulated liposomes (bright spots) with the macrophages (arrows) in the plaque. Image C is the corresponding bright-field image.

FIG. 3 shows representative fluorescence microscopy images of plaque sections obtained from an LDb mouse that was injected with rhodamine-associated liposomes. Image A demonstrates the staining of macrophages for F4/80 antigen (darkened region); the cell nucleus is counterstained with hematoxylin. Image B demonstrates the localization of rhodamine-associated liposomes (bright spots) in the plaque. Image C demonstrates the staining of the corresponding section of the cell nucleus with 4′-6-Diamidino-2-phenylindole (DAPI), and is merged with the rhodamine image (Image B).

FIG. 4 shows representative fluorescence microscopy images of plaque sections obtained from an LDb mouse that was injected with rhodamine-associated liposomes. Image A demonstrates the staining of macrophages for F4/80 antigen (darkened region); the cell nucleus is counterstained with hematoxylin. Image B demonstrates the localization of rhodamine-associated liposomes (bright spots) in the plaque. Image C demonstrates the staining of the corresponding section of the cell nucleus with DAPI, and is merged with the rhodamine image (Image B).

FIG. 5 shows representative fluorescence microscopy images of plaque sections obtained from an LDb mouse that was injected with phosphate buffered saline (negative control). Image A demonstrates the staining of macrophages for F4/80 antigen; the cell nucleus is counterstained with hematoxylin. Image B demonstrates the auto-fluorescence signal (background) in the plaque. Image C demonstrates the staining of the corresponding section of the cell nucleus with DAPI, and is merged with Image B.

### DETAILED DESCRIPTION

The development of atherosclerotic plaques proceeds by, for example, the localization of macrophage cells in a site of inflammation surrounding the so called “fatty streak” of deposited lipids on the walls of a major artery. Imaging agents that are localized into these macrophages enable the visualization of the plaque.

Liposomal compositions and methods are provided for imaging, detecting, and evaluating macrophages, e.g., activated macrophages, and vascular plaque, e.g., vulnerable plaque. Vulnerable plaques contain macrophages, e.g., activated macrophages, which accumulate on arterial walls. In one embodiment, the liposomal compositions are taken up by macrophages, e.g., activated macrophages. Therefore, visualization of the plaque containing the macrophages is possible using routine imaging technology, such as, by x-ray imaging, ultrasonagraphy, computed tomography (CT), computed tomography angiography (CTA), electron beam (EBT), magnetic resonance imaging (MRI), magnetic resonance angiography (MRA), positron emission tomography, and other imaging technologies.

When administered to a subject, the liposomal compositions remain substantially confined to the intravascular space and, therefore, do not significantly permeate to the interstitial space or extrastitial fluids, thus facilitating the imaging of blood pools and vascular structures, e.g., vascular tissue, vascular beds, and organ tissues, as well as plaque, such as vulnerable plaque and macrophages. Furthermore, the liposomal compositions are excreted from the body via the liver rather than, for example, the renal system, and, therefore, remain in the body for a longer period of time than contrast agents that are excreted via the renal system.

Some embodiments disclosed herein feature liposomal compositions that remain in the vascular structures for an extended period of time at functionally active concentrations with a half-life of about 18 hours until the contrast agent is metabolized by the liver. As such, multiple images may be taken after a single, low-dose administration of the liposomal compositions. Furthermore, this functional half-life time is long enough to allow vascular scanning in vascular beds of interest (kidney, liver, heart, brain and elsewhere) to be performed. This is in contrast to agents currently in use which diffuse quickly, e.g., after several seconds or minutes, allowing only a small window of time to perform imaging following administration of the agent. Furthermore, because the liposomal compositions are substantially confined to the vascular space, whole body vascular imaging, as well as imaging of whole body plaque burden, is allowed using routine imaging technology known to those of skill in the art, e.g., x-ray imaging, ultrasonagraphy, computed tomography (CT), computed tomography angiography (CTA), electron beam (EBT), magnetic resonance imaging (MRI), magnetic resonance angiography (MRA), and positron emission tomography. In addition, the minimal diffusion of the liposomal compositions from the intravascular space allows imaging of areas of vascular disease or disorder, or vascular damage, e.g., leakage, tissue damage, or tumors, to be visualized due to the accumulation of the contrast agent in areas outside of the intravascular space.

The terms “vasculature,” “vessels,” and “circulatory system” are intended to include any vessels through which blood circulates, including, but not limited to veins, arteries, arterioles, venules, and capillaries.

The term “vascular disease or disorder,” also commonly referred to as “cardiovascular disease,” “coronary heart disease” (CHD), and “coronary artery disease” (CAD) as used herein, refers to any disease or disorder effecting the vascular system, including the heart and blood vessels. A vascular disease or disorder includes any disease or disorder characterized by vascular dysfunction, including, for example, intravascular stenosis (narrowing) or occlusion (blockage) due to, for example, a build-up of plaque on the inner arterial walls, and diseases and disorders resulting therefrom.

The term “thrombotic or thromboembolic event” includes any disorder that involves a blockage or partial blockage of an artery or vein with a thrombosis. A thrombic or thrombolic event occurs when a clot forms and lodges within a blood vessel which may occur, for example, after a rupture of a vulnerable plaque. Examples of vascular diseases and disorders include, without limitation, atherosclerosis, CAD, MI, unstable angina, acute coronary syndrome, pulmonary embolism, transient ischemic attack, thrombosis (e.g., deep vein thrombosis, thrombotic occlusion and re-occlusion and peripheral vascular thrombosis), thromboembolism, e.g., venous thromboembolism, ischemia, stroke, peripheral vascular diseases, and transient ischemic attack.

As used herein, the term “plaque,” also commonly referred to as “atheromas,” refers to the substance which builds up on the inner surface of the vessel wall resulting in the narrowing of the vessel and is the common cause of CAD. Usually, plaque comprises fibrous connective tissue, lipids (fat) and cholesterol. Frequently, deposits of calcium salts and other residual material may also be present. Plaque build-up results in the erosion of the vessel wall, diminished elasticity (stretchiness) of the vessel, and eventual interference with blood flow. Blood clots may also form around the plaque deposits, thus further interfering with blood flow. Plaque stability is classified into two categories based on the composition of the plaque. As used herein, the term “stable” or “inactive” plaques refers to those which are calcified or fibrous and do not present a risk of disruption or fragmentation. These types of plaques may cause anginal chest pain but rarely myocardial infarction in the subject. Alternatively, the term “vulnerable” or “active” plaque refers to those comprising a lipid pool covered with a thin fibrous cap. Within the fibrous cap is a dense infiltrate of smooth muscle cells, macrophages, foam cells, and lymphocytes. Vulnerable plaques may not block arteries but may be ingrained in the arterial wall, so that they are undetectable and may be asymptomatic. Furthermore, vascular plaques are considered to be at a high risk of disruption. Disruption of the vulnerable plaque is a result of intrinsic and extrinsic factors, including biochemical, haemodynamic, and biomechanical stresses resulting, for example, from blood flow, as well as inflammatory responses from such cells as, for example, macrophages.

As used herein, the term “macrophage” refers to the relatively long-lived phagocytic cell of mammalian tissues, derived from blood monocytes. Macrophages are involved in all stages of immune responses. Macrophages play an important role in the phagocytosis (digestion) of foreign bodies, such as bacteria, viruses, protozoa, tumor cells, cell debris, and the like, as well as the release of chemical substances, such as cytokines, growth factors, and the like, that stimulate other cells of the immune system. Macrophages are also involved in antigen presentation as well as tissue repair and wound healing. There are many types of macrophages, including alveolar and peritoneal macrophages, tissue macrophages (histiocytes), Kupffer cells of the liver, and osteoclasts of the bone, all of which are within the scope of the invention. Macrophages may also further differentiate within chronic inflammatory lesions to epitheliod cells or may fuse to form foreign body giant cells (e.g., granulomas) or Langerhan giant cells.

A typical liposomal composition comprises a lipid or phospholipid, a stabilizing excipient such as cholesterol, and a polymer-derivatized phospholipid. Suitable examples of lipids or phospholipids, stabilizing excipients, and polymer-derivatized phospholipids are set forth in, for example, U.S. patent application Ser. Nos. 10/830,190, 11/595,808, and 11/568,936, all of which are incorporated by reference in their entireties herein.

The liposomal compositions typically encapsulate or associate a contrast agent. It should be noted that for purposes of the present application, the identity of the contrast agent is not of substantial importance. Rather, the liposome composition (e.g., cholesterol; at least one phospholipid; and at least one phospholipid which is derivatized with a polymer chain) and the small size (e.g., less than 150 nm, as described below) provide the desired localization. In other words, for purposes of the present invention, the liposomal compositions will perform (mechanistically speaking) identically regardless of the contrast agent used. Nonetheless, suitable contrast agents include, for example, fluorescent dyes, such as, for example, fluorescein iso-thiocynate (FITC) and rhodamine; CT contrast agents including iodinated compounds such as iohexol, iodixanol, and iotrolan, and as otherwise described in U.S. patent application Ser. Nos. 10/830,190, 11/595,808, and 11/568,936; and MRI contrast agents including lanthanide aminocarboxylate complexes such as Gadolinium (III) DTPA, Gd-DOTA, Gd-DOTAP, and Gd-DOTMA.

The liposomes are typically approximately 100 nm in average diameter, but may range from about 50 to about 150 nm in average diameter. Thus, a suitable liposome average diameter may be less than 150 nm, less than 120 nm, and less than 100 nm.

The liposome agents may be prepared, for example, by the methods disclosed in U.S. patent application Ser. Nos. 10/830,190, 11/595,808, and 11/568,936.

In one embodiment, the at least one first lipid or phospholipid is present in the amount of about 55 to about 75 mol %; the at least one second lipid or phospholipid which is derivatized with one or more polymers is present in the amount of about 1 to about 20 mol %; and the at least one sterically bulky excipient is present in the amount of about 25 to about 50 mol %.

In another embodiment, the at least one first lipid or phospholipid is present in the amount of about 55 mol %; the at least one second lipid or phospholipid which is derivatized with one or more polymers is present in the amount of about 5 mol %; and the at least one sterically bulky excipient is present in the amount of about 40 mol %.

### EXAMPLES

A lipid mixture comprising 1,2-dipalmitoyl-sn-glycero-3-phosphocholine (DPPC), cholesterol, and 1,2-distearoyl-sn-glycero-3-phosphoethanolamine-N-[methoxy(poly(ethylene glycol))-2000] (mPEG2000-DSPE) in the ratio 55:40:5 was dissolved in ethanol at 60° C. This lipid solution was mixed with a 2 mM fluorescein iso-thiocynate (FITC) solution and stirred for 2 hr at 60° C. The FITC is encapsulated by the liposomes. Subsequently, the solution was sequentially extruded at 60° C. through a high-pressure extruder with seven passes through a 200 nm Nuclepore filter membrane and ten passes through a 100 nm Nuclepore membrane. The resulting solution was diafiltered using a MicroKros module of 500 kDa molecular weight cut-off to remove unencapsulated FITC molecules to yield the FITC-encapsulated liposomal agent.

Six apoliprotein E knockout (ApoE −/−) mice (27-32 gm) were used for the study. Four mice were used for the FITC-encapsulated liposome agent. Two mice were used for the control group (injected with saline buffer). The animals were anesthetized with a 5% isoflurane solution to render them unconscious and were maintained on 2% isoflurane and oxygen to facilitate injection of liposomes and draw blood. Subsequently, the FITC-encapsulated liposomal agent (0.1 μmoles of lipid per gram of body weight) was injected intravenously via the tail vein. Blood samples were drawn via the tail vein at 1, 2, 4, 8, and 24 hour time periods. After 24 hours, the animal was anesthetized with 5% isoflurane, treated with 100 uL of heparin-sodium (porcine derived, 1000 IU/ml), and sacrificed via bleeding of the carotid artery. The aorta was dissected, cleaned, and placed in boats containing OCT. The boats were then cut into blocks and embedded in paraffin and stored at −80° C. The aortas were sectioned and the cell nucleus was stained with hematoxylin. The macrophages were stained with F4/80 antigen (MCA497, Serotec). Adjacent unstained aorta sections were used for imaging the presence of FITC-encapsulated liposomes in plaque.

Fluorescence imaging of the aorta sections was performed to demonstrate the localization of liposomal agent (in this case, FITC-encapsulated liposomal agent) and macrophages in atherosclerotic plaque lesions.

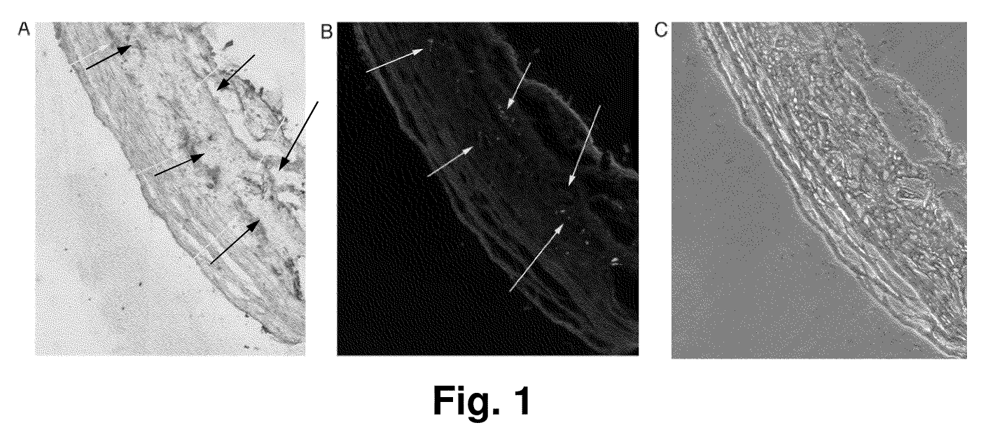

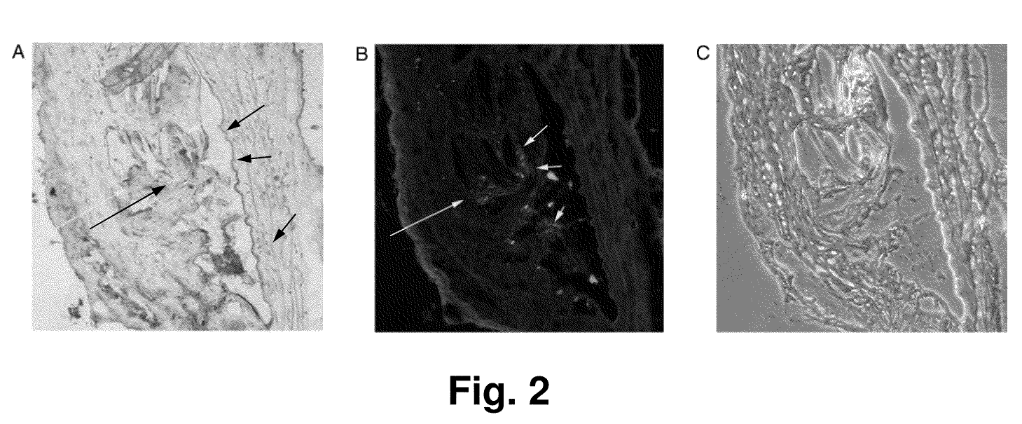

Immunostaining with F4/80 antigen clearly demonstrated the localization of macrophages in atherosclerotic lesions (FIGS. 1A and 2A). FITC-encapsulated liposomes were also visibly co-localized in areas of macrophage content in the plaque (FIGS. 1B and 2B).

FIG. 1 shows representative fluorescence microscopy images of plaque sections obtained from an ApoE −/− mouse that was injected with fluorescein iso-thiocynate (FITC)-encapsulated liposomes. Image A demonstrates the staining of macrophages for F4/80 antigen (darkened regions); the cell nucleus is counterstained with hematoxylin. Image B demonstrates the co-localization of FITC-encapsulated liposomes (bright spots) with the macrophages (arrows) in the plaque. Image C is the corresponding bright-field image.

FIG. 2 shows representative fluorescence microscopy images of plaque sections obtained from an ApoE −/− mouse that was injected with FITC-encapsulated liposomes. Image A demonstrates the staining of macrophages for F4/80 antigen (darkened region); the cell nucleus is counterstained with hematoxylin. Image B demonstrates the co-localization of FITC-encapsulated liposomes (bright spots) with the macrophages (arrows) in the plaque. Image C is the corresponding bright-field image.

In a second illustration, a different contrast agent, the fluorescent dye rhodamine, was used for the preparation of liposomes. Rhodamine is “associated” with the liposomes, rather than “encapsulated” within the liposomes, in the sense that rhodamine is attached to a lipid and inserted in the liposome bilayer (shell). A lipid mixture comprising 1,2-dipalmitoyl-sn-glycero-3-phosphocholine (DPPC), cholesterol, 1,2-distearoyl-sn-glycero-3-phosphoethanolamine-N-[methoxy(poly(ethylene glycol))-2000] (mPEG2000-DSPE) and lissamine rhodamine B 1,2-dihexadecanoyl-sn-glycero-3-phosphoethanolamine (rhodamine DHPE) in the ratio 55:40:4.7:0.3 was dissolved in ethanol at 60° C. This lipid solution was mixed with a 150 mM sodium chloride solution and stirred for 2 hr at 60° C. The solution was sequentially extruded at 60° C. through a high-pressure extruder with seven passes through a 200 nm Nuclepore filter membrane and ten passes through a 100 nm Nuclepore membrane.

Three LDb (LDLR−/−Apobec1−/−) mice (27-32 gm) were used for the study. Two mice were used for the rhodamine-liposomal agent. One mouse was used for control group (injected with phosphate buffered saline). The animals were anesthetized with a 5% isoflurane solution to render them unconscious and were maintained on 2% isoflurane and oxygen to facilitate injection of liposomes. Subsequently, the rhodamine-liposomal agent (0.1 μmoles of lipid per gram of body weight) was injected intravenously via the tail vein. After 7 days, the animal was anesthetized with 5% isoflurane, treated with 100 uL of heparin-sodium (porcine derived, 1000 IU/ml), and sacrificed via bleeding of the carotid artery. The aorta was dissected, cleaned, and placed in 10% formalin in buffered saline. The aortas were then cut into pieces and paraffin embedded. The paraffin embedded aortas were sectioned on to glass slides for further processing. The cell nucleus was stained with hematoxylin and the macrophages were stained with F4/80 antigen (MCA497, Serotec). Adjacent unstained aorta sections were used for imaging the presence of rhodamine-liposomes in plaque. For the fluorescence microscopy, cell nucleus was also stained using DAPI.

Fluorescence microscopy of the aorta sections was performed to demonstrate the localization of liposomal agent (in this case, rhodamine-associated liposomal agent) and macrophages in atherosclerotic plaque lesions.

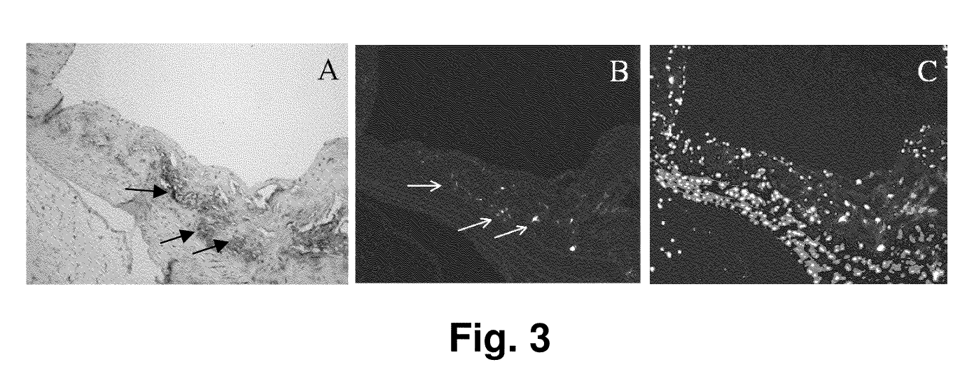

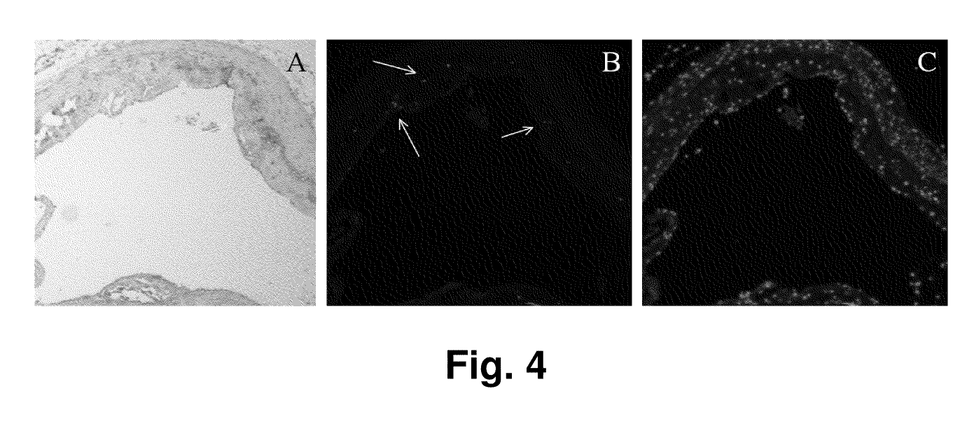

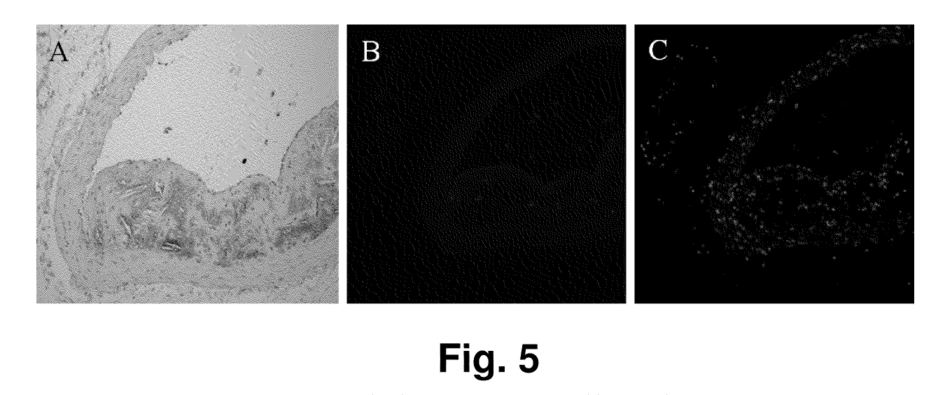

Immunostaining with F4/80 antigen clearly demonstrated the localization of macrophages in atherosclerotic lesions (FIGS. 3A, 4A, and 5A). Rhodamine-liposomes were also visibly co-localized in areas of macrophage content in the plaque (FIGS. 3B and 4B). Very little auto-fluorescence signal was observed in the sections obtained from a non-treated mouse (FIG. 5B) as indicated by the low spot intensity in the image.

FIG. 3 shows representative fluorescence microscopy images of plaque sections obtained from an LDb mouse that was injected with rhodamine-liposomes. Image A demonstrates the staining of macrophages for F4/80 antigen (darkened region); the cell nucleus is counterstained with hematoxylin. Image B demonstrates the localization of rhodamine-liposomes (bright spots) in the plaque. Image C demonstrates the staining of the corresponding section of the cell nucleus with DAPI, and is merged with the rhodamine image (Image B).

FIG. 4 shows representative fluorescence microscopy images of plaque sections obtained from an LDb mouse that was injected with rhodamine-liposomes. Image A demonstrates the staining of macrophages for F4/80 antigen (darkened region); the cell nucleus is counterstained with hematoxylin. Image B demonstrates the localization of rhodamine-liposomes (bright spots) in the plaque. Image C demonstrates the staining of the corresponding section of the cell nucleus with DAPI, and is merged with the rhodamine image (Image B).

FIG. 5 shows representative fluorescence microscopy images of plaque sections obtained from an LDb mouse that was injected with phosphate buffered saline (negative control). Image A demonstrates the staining of macrophages for F4/80 antigen; the cell nucleus is counterstained with hematoxylin. Image B demonstrates the auto-fluorescence signal (background) in the plaque. Image C demonstrates the staining of the corresponding section of the cell nucleus with DAPI, and is merged with Image B.

It is, of course, not possible to describe every conceivable combination of components or methodologies for purposes of describing the compositions, methods, and so on provided herein. Additional advantages and modifications will readily appear to those skilled in the art. Therefore, the invention, in its broader aspects, is not limited to the specific details and illustrative examples shown and described. Accordingly, departures may be made from such details without departing from the spirit or scope of the applicants' general inventive concept. A person of ordinary skill will readily recognize that optimizing or manipulating any one of these variables may or will require or make possible the manipulation of one or more of the other of these variables, and that any such optimization or manipulation is within the spirit and scope of the present embodiments.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. It should be noted that the term “about” may mean up to and including ±10% of the stated value. For example, “about 10” may mean from 9 to 11.

Furthermore, while the compositions, methods, and so on have been illustrated by describing examples, and while the examples have been described in considerable detail, it is not the intention of the applicant to restrict, or in any way, limit the scope of the appended claims to such detail. Thus, this application is intended to embrace alterations, modifications, and variations that fall within the scope of the appended claims. The preceding description is not meant to limit the scope of the invention. Rather, the scope of the invention is to be determined by the appended claims and their equivalents.

Finally, to the extent that the term “includes” or “including” is employed in the detailed description or the claims, it is intended to be inclusive in a manner similar to the term “comprising,” as that term is interpreted when employed as a transitional word in a claim. Furthermore, to the extent that the term “or” is employed in the claims (e.g., A or B) it is intended to mean “A or B or both.” When the applicants intend to indicate “only A or B, but not both,” then the term “only A or B but not both” will be employed. Similarly, when the applicants intend to indicate “one and only one” of A, B, or C, the applicants will employ the phrase “one and only one.” Thus, use of the term “or” herein is the inclusive, and not the exclusive use. See Bryan A. Garner, A Dictionary of Modern Legal Usage 624 (2d. Ed. 1995).

What is claimed is:

1. A method for imaging atherosclerotic plaques, the method comprising:
introducing a composition into a subject's vasculature, the composition comprising:
liposomes, the liposomes encapsulating one or more nonradioactive contrast enhancing agents, and the liposomes comprising:
cholesterol;
at least one phospholipid; and
at least one phospholipid which is derivatized with a polymer chain,
wherein the average diameter of the liposomes is less than 150 nanometers;
generating images of the subject's vasculature; and
analyzing the images to detect and/or evaluate an atherosclerotic plaque in the subject.

2. The method of claim 1, wherein the at least one phospholipid comprises 1,2-dipalmitoyl-sn-glycero-3-phosphocholine (DPPC).

3. The method of claim 1, wherein the at least one phospholipid which is derivatized with a polymer chain comprises 1,2-distearoyl-sn-glycero-3-phosphoethanolamine-N-[methoxy(poly(ethylene glycol))-2000] (mPEG2000-DSPE).

4. The method of claim 1, wherein the at least one phospholipid is present in the amount of about 55 to about 75 mol %; the at least one phospholipid which is derivatized with a polymer chain is present in the amount of about 1 to about 20 mol %; and the cholesterol is present in the amount of about 25 to about 50 mol %.

5. The method of claim 1, wherein the at least one phospholipid is present in the amount of about 55 mol %; the at least one phospholipid which is derivatized with a polymer chain is present in the amount of about 5 mol %; and the cholesterol is present in the amount of about 40 mol %.

6. The method of claim 1, wherein the liposomes have an average diameter of less than about 120 nm.

7. The method of claim 1, wherein the liposomes have an average diameter of less than or equal to about 100 nm.

8. The method of claim 1, wherein the generating images comprises generating X-ray images.

9. The method of claim 1, wherein the generating images comprises generating images before and after introducing the composition into the subject's vasculature.

10. The method of claim 1, wherein the analyzing the images comprises distinguishing areas having an enhanced signal from areas having little or no signal.

11. The method of claim 1, wherein the composition is characterized in that the composition accumulates in an atherosclerotic plaque of the subject's vasculature, in comparison to an area not comprising an atherosclerotic plaque, thereby enhancing the signal in the atherosclerotic plaque.

12. The method of claim 1, wherein the generating images comprises generating X-ray images using at least one of computed tomography, micro-computed tomography, mammography, and chest X-ray.

13. The method of claim 1, wherein the generating images comprises generating images using at least one of MRI, ultrasound, and optical imaging, including fluorescence or bioluminescence imaging.

14. A method for imaging atherosclerotic plaques in a subject, the method comprising:
administering a liposomal composition comprising liposomes to the subject, the liposomes comprising:
at least one first lipid or phospholipid;
at least one second lipid or phospholipid which is derivatized with one or more polymers; and
at least one sterically bulky excipient capable of stabilizing the liposomes; and wherein the liposomes:
(1) encapsulate a non-radioactive contrast enhancing agent in a concentration of about 130-200 mg of non-radioactive contrast enhancing agent per mL of liposomal composition; and
(2) have an average diameter of less than 150 nm;
generating images of the subject's vasculature; and
analyzing the images to detect and/or evaluate an atherosclerotic plaque in the subject.

15. The method of claim 14, wherein the generating images comprises generating X-ray images.

16. The method of claim 14, wherein the generating images comprises generating images before and after administering the liposomal composition to the subject.

17. The method of claim 14, wherein the analyzing the images comprises distinguishing areas having an enhanced signal from areas having little or no signal.

18. The method of claim 14, wherein the liposomal composition is characterized in that the liposomal composition accumulates in an atherosclerotic plaque of the subject's vasculature, in comparison to an area not comprising an atherosclerotic plaque, thereby enhancing the signal in the atherosclerotic plaque.

19. The method of claim 14, wherein the generating images comprises generating X-ray images using at least one of computed tomography, micro-computed tomography, mammography, and chest X-ray.

20. The method of claim 14, wherein the generating images comprises generating images using at least one of MRI, ultrasound, and optical imaging, including fluorescence or bioluminescence imaging.
